(19) United States
(12) Patent Application Publication
(10) Pub. No.: US 2025/0114344 A1
(43) Pub. Date: Apr. 10, 2025

(54) COMBINATORIAL PHARMACOTHERAPY FOR THE RESTORATION OF NEURO-FUNCTION AND COMBINATORIAL PHARMACOLOGICAL COMPOSITION THEREFORE

(71) Applicant: Neuro-Innovators, LLC., Sewickley, PA (US)

(72) Inventors: Peter C. Doyle, Sherborn, MA (US); John Howison Schroeder, Sewickley, PA (US); Ian Jeffery Reynolds, Leefdaal (BE); Christopher J. Doyle, West Lebanon, NH (US); Florenta Aura Kullmann, Pittsburgh, PA (US); Mark Andrew Cochran, Ruckersville, VA (US)

(21) Appl. No.: 18/922,226

(22) Filed: Oct. 21, 2024

Related U.S. Application Data
(63) Continuation of application No. PCT/US23/19320, filed on Apr. 20, 2023.
(60) Provisional application No. 63/332,957, filed on Apr. 20, 2022.

(51) Int. Cl. A61K 31/4709; A61K 31/155; A61K 31/4184

(52) U.S. Cl. CPC A61K 31/4709; A61K 31/155; A61K 31/4184

(57) ABSTRACT

A pharmacotherapeutic protocol enhancing neuro-restoration in the human brain for patients suffering with brain injuries and diseases, wherein the protocol comprises treating the patient with a combinatorial pharmacological composition comprising a pharmacologically effective amount of a first Neuroplasticity PA therapeutic agent exhibiting a mechanism of plasticity in at least one of Neurogenesis, Synaptogenesis, Remyelination, Angiogenesis, Bioenergetics, Inflammation and Apoptosis, and a pharmacologically effective amount of a second Neuroplasticity PA therapeutic agent exhibiting a mechanism of plasticity in at least one of Neurogenesis, Synaptogenesis, Remyelination, Angiogenesis, Bioenergetics, Inflammation and Apoptosis; wherein the mechanism of plasticity of the first Neuroplasticity PA therapeutic agent differs from the mechanism of plasticity of the second Neuroplasticity PA therapeutic agent. Combinatorial pharmacological composition are also disclosed.

### RELATED APPLICATIONS

The present application is a continuation of International Patent Application Serial Number PCT/US2023/019320 filed Apr. 20, 2023 titled “Combinatorial Pharmacotherapy for the Restoration of Neuro-Function and Combinatorial Pharmacological Composition Therefore” and published Oct. 26, 2023 as publication WO 2023/205369 which application and publication are incorporated herein by reference.

International Patent Application Serial Number PCT/US2023/019320claims the benefit of U.S. provisional patent application Ser. No. 63/332,957 filed Apr. 20, 2022 titled “Combinatorial Pharmacotherapy for the Restoration of Neuro-Function and Combinatorial Pharmacological Composition Therefore.”

### BACKGROUND OF THE INVENTION

### 1. FIELD OF THE INVENTION

This invention generally relates to enhancing neuro-restoration or the building of new neurons, axons, and synapses and remyelination in the human brain by combining two or more pharmacologic molecules in a pharmacotherapeutic protocol.

### 2. BACKGROUND INFORMATION

As reported in the May 1, 2019 journal Neurology out-of-pocket costs are rapidly on the rise for commonly prescribed neurologic medications. This is of great significance as it has been estimated that one in six people lives with a neurologic disease or disorder. It has further been estimated in 2019 that the annual cost of treating neurologic disorders in the United States is more than $500 billion (with the current cost expected to be substantially higher).

Aside from cost to patients there are extraneous stresses on the economy from brain disorders and diseases. It is estimated that brain disorders and diseases cost U.S. economy over $2 trillion annually with these costs underscoring the scale of the need and opportunity for greater research and innovative new treatments to improve health and drive prosperity. See for earlier estimates Information Technology & Innovation Foundation Jul. 11, 2016.

Unfortunately, the field of commercial pharmacology and drug development is, generally, focused on the development of single molecules addressing a single mechanism of action to provide comprehensive therapeutics to cure a disease. This strategy has often proven to be a failure, particularly in the neurosciences.

Success of a pharmacological agent for neurologic use is limited by its capacity to cross the blood brain barrier (BBB) in sufficient volume to produce the necessary effect. An effective, high functioning agent is often rendered clinically ineffective due the inability to pass in a therapeutically sufficient quantity across the blood brain barrier, and crossing this barrier becomes the governing factor of an agent's clinical and commercial success.

The present invention relates to combinatorial pharmacological compositions. A “combinatorial pharmacological composition” is defined herein as the combination of two or more distinct therapeutic agents into a single composition. The PHARNEXT brand agent illustrates synergistic therapeutic benefits of two drug delivered simultaneously to cure or slow the progression of a genetic disorder known as Charcot Marie Tooth (technically Charcot Marie Tooth is a group of inherited disorders which cause nerve damage mostly in arms and legs. Weakness in limbs, hammer toes, and loss of sensation in limbs are the common symptoms). Currax Pharmaceuticals' CONTRAVE brand drug is naltrexone HCL and bupropion HCL extended release was approved in 2014 by the FDA and has become a popular obesity medication. The CONTRAVE brand drug works in two parts of the brain to help some adults control their eating, resulting in sustained weight loss. There are other examples of new FDA indications using two drugs or molecules concurrently to form a combinatorial pharmacological composition, but they remain uncommon.

“Neuroplasticity” is defined herein as the capacity of the brain to rebuild, rewire, and recover after impairments from injury or disease. “Neuro-restoration” is defined herein as the rebuilding of neurons, axons, and/or synapses and/or remyelination in the human brain. “Combinatorial pharmacotherapy” is defined herein as a pharmacotherapeutic protocol implementing at least one combinatorial pharmacological composition.

Some of the inventors of the present application developed a neurologic rehabilitation and training method utilizing oculomotor, visual and vestibular tasks on subjects with pharmacologically induced neuroplasticity set forth in U.S. Patent Publication 2021-0330255, which represents useful background to the present application.

There remains a great need for enhancing neuro-restoration in the human brain by effective pharmacotherapeutic protocols.

### SUMMARY OF THE INVENTION

One aspect of the present invention provides a pharmacotherapeutic protocol enhancing neuro-restoration in the human brain for patients suffering with brain injuries and diseases, wherein the protocol comprises treating the patient with a combinatorial pharmacological composition comprising a pharmacologically effective amount of a first Neuroplasticity PA therapeutic agent exhibiting a mechanism of plasticity in at least one of Neurogenesis, Synaptogenesis, Remyelination, Angiogenesis, Bioenergetics, Inflammation and Apoptosis, and a pharmacologically effective amount of a second Neuroplasticity PA therapeutic agent exhibiting a mechanism of plasticity in at least one of Neurogenesis, Synaptogenesis, Remyelination, Angiogenesis, Bioenergetics, Inflammation and Apoptosis; wherein the mechanism of plasticity of the first Neuroplasticity PA therapeutic agent differs from the mechanism of plasticity of the second Neuroplasticity PA therapeutic agent. The phrase “Neuroplasticity PA therapeutic agent” is detailed further below, but is defined herein as a therapeutic agent that has proven neuroplasticity effects and has been approved by an appropriate regulatory agency for medical use in human patients.

The pharmacotherapeutic protocol according to the present invention may provide specifically for patient who is suffering from i) an injury to the central nervous system comprising at least one of stroke, TBI, PTSD, CTE and Spinal Cord Injury; ii) from at least one of dementia and movement disorders comprising at least one of Alzheimer's disease, Parkinson's disease and ALS.; iii) from at least one of headaches and depression comprising at least one of migraine, depression and Bipolar disorder; iv) from an immune system attack comprising at least one of MS, Muscular Dystrophy, Rheumatoid Arthritis and Lupus; v) from a neurological or psychological disorder comprising at least one of Cerebral Palsy, Schizophrenia, Epilepsy, Autism, and Dyslexia; and vi) from a cognitive deficit comprising at least one of long COVID, Cancer Chemo Fog, Anesthetic fog, Chronic fatigue and Fibromyalgia.

In one embodiment of the invention the first Neuroplasticity PA therapeutic agent comprises one of Telmisartan, Metformin and Cilostazol, and the second Neuroplasticity PA therapeutic agent comprises one of Telmisartan, Metformin and Cilostazol and which differs from the first Neuroplasticity PA therapeutic agent, and wherein the combinatorial pharmacological composition further includes a third Neuroplasticity PA therapeutic agent.

In one embodiment of the invention the mechanism of plasticity of the first Neuroplasticity PA therapeutic agent and the mechanism of plasticity of the second Neuroplasticity PA therapeutic agent both includes reducing Inflammation. In one embodiment of the invention the mechanism of plasticity of the first Neuroplasticity PA therapeutic agent and the mechanism of plasticity of the second Neuroplasticity PA therapeutic agent both includes enhancing Neurogenesis. In one embodiment of the invention the mechanism of plasticity of the first Neuroplasticity PA therapeutic agent and the mechanism of plasticity of the second Neuroplasticity PA therapeutic agent both includes enhancing Angiogenesis. In one embodiment of the invention the mechanism of plasticity of the first Neuroplasticity PA therapeutic agent and the mechanism of plasticity of the second Neuroplasticity PA therapeutic agent both includes enhancing Remyelination. In one embodiment of the invention the mechanism of plasticity of the first Neuroplasticity PA therapeutic agent and the mechanism of plasticity of the second Neuroplasticity PA therapeutic agent both includes enhancing Bioenergetics.

The pharmacotherapeutic protocol according to one embodiment of the invention provides wherein the combinatorial pharmacological composition further includes at least one pharmacological psychedelic which has been shown to promote neuroplasticity comprising at least one of tryptamines, amphetamines and ergoline.

One aspect of the present invention provides a Combinatorial pharmacological composition enhancing neuro-restoration in the human brain for patients suffering with brain injuries and diseases, wherein the combinatorial pharmacological composition comprises a pharmacologically effective amount of a first Neuroplasticity PA therapeutic agent exhibiting a mechanism of plasticity in at least one of Neurogenesis, Synaptogenesis, Remyclination, Angiogenesis, Bioenergetics, Inflammation and Apoptosis, and a pharmacologically effective amount of a second Neuroplasticity PA therapeutic agent exhibiting a mechanism of plasticity in at least one of Neurogenesis, Synaptogenesis, Remyclination, Angiogenesis, Bioenergetics, Inflammation and Apoptosis; wherein the mechanism of plasticity of the first Neuroplasticity PA therapeutic agent differs from the mechanism of plasticity of the second Neuroplasticity PA therapeutic agent.

One aspect of the invention provides a combinatorial pharmacotherapy enhancing neuro-restoration, or building of new neurons, axons, and synapses and remyelination in the human brain, by implementing a combinatorial pharmacological composition combining two or more pharmacologic molecules in a pharmacotherapeutic protocol wherein the two or more pharmacologic molecules concurrently moderates/manages the function of two or more mechanisms of action necessary for enabling the creation of neurons, axons, synapses, and or remyelination to enhance the environment for and or accelerate the growth of neuronal networks damaged by or malfunctioning as a result of disease or trauma.

One aspect of the invention may further include the addition of molecules that increase the flow of blood, and all materials contained in the blood, to the brain essentially increasing permeability of the blood brain barrier or which facilitate transport with liposomes or receptor targeted carriers.

These and other advantages of the present invention will be clarified in the following description taken together with the following figures.

### BRIEF DESCRIPTION OF THE FIGURES

FIG. 1 schematically illustrates the mechanism of plasticity of a Neuroplasticity PA therapeutic agent forming a combinatorial pharmacological composition enhancing neuro-restoration in the human brain for patients suffering with brain injuries and diseases according to one representative example of the present invention;

FIG. 2 schematically illustrates the mechanisms of plasticity of three Neuroplasticity PA therapeutic agents forming the combinatorial pharmacological composition of FIG. 1; and

FIG. 3 schematically illustrates the mechanism of plasticity of the combinatorial pharmacological composition of FIG. 1.

### DESCRIPTION OF THE PREFERRED EMBODIMENTS

The present invention establishes a framework and method for engineering drugs, namely combinatorial pharmacological compositions 100, to accelerate brain neuroplasticity for patients suffering with brain injuries and diseases. Neuroplasticity is an untapped and critical component to combatting brain impairments related to dementia, depression, movement disorders, immune-system attacks, psychiatric illness, and cognition deficits. These ailments place a heavy burden on individuals, family, communities, nations, and the globe. Neuroplasticity itself is an emerging arm of clinical neuroscience, supported by today's greater computing power, innovations in imaging technology, miniaturization in devices for testing and monitoring, and advances in genetics, proteomics, molecular biology, chemistry, and physics. As detailed below the present invention yields a framework for engineering combinatorial pharmacological compositions 100 to help repair major brain impairments. As discussed below, with the unique and complex nature of each human brain, specific combinatorial pharmacological compositions addressing multiple mechanism of action (e.g., not a single molecule) are critical to successful improvements in patient outcomes. The combinatorial pharmacological compositions 100 of the present invention are designed to promote cellular growth and repair, while simultaneously inhibiting inflammation and cell death.

As discussed below the present invention provides pharmacotherapeutic protocol enhancing neuro-restoration in the human brain for patients suffering with brain injuries and diseases, wherein the protocol comprises treating the patient with a combinatorial pharmacological composition 100 comprising a pharmacologically effective amount of a first Neuroplasticity PA therapeutic agent 30 exhibiting a mechanism of plasticity 32 in at least one of Neurogenesis 12, Synaptogenesis 14, Remyelination 16, Angiogenesis 20, Bioenergetics 18, Inflammation 22 and Apoptosis 24, and a pharmacologically effective amount of a second Neuroplasticity PA therapeutic agent 40 exhibiting a mechanism of plasticity 42 in at least one of Neurogenesis 12, Synaptogenesis 14, Remyelination 16, Angiogenesis 20, Bioenergetics 18, Inflammation 22 and Apoptosis 24; wherein the mechanism of plasticity 32 of the first Neuroplasticity PA therapeutic agent 30 differs from the mechanism of plasticity 42 of the second Neuroplasticity PA therapeutic agent 40. As detailed below the combinatorial pharmacological composition 100 may further include a pharmacologically effective amount of a third (or more) Neuroplasticity PA therapeutic agent 50 exhibiting a mechanism of plasticity 52 in at least one of Neurogenesis 12, Synaptogenesis 14, Remyelination 16, Angiogenesis 20, Bioenergetics 18, Inflammation 22 and Apoptosis 24. The mechanism of plasticity 52 of the third Neuroplasticity PA therapeutic agent 50 will differ from the mechanisms of plasticity 32 and 42.

The preferred embodiments of the present invention restores neurofunction damaged or lost as a result of disease or trauma. For a general discussion of neuro-restoration see Azad T D, Veeravagu A, Steinberg G K. Neurorestoration after stroke. Neurosurg Focus. 2016 May; 40(5):E2. doi: 10.3171/2016.2.FOCUS15637. PMID: 27132523; PMCID: PMC4916840.

The various embodiments and examples of the present invention as presented herein are understood to be illustrative of the present invention and not restrictive thereof and are non-limiting with respect to the scope of the invention.

As noted above the phrase “Neuroplasticity PA therapeutic agent” is defined herein as a therapeutic agent that has proven neuroplasticity effects and has been approved by an appropriate regulatory agency for medical use in human patients. Proven neuroplasticity effects of an agent is defined herein as neuroplasticity effects of an agent documented in at least one peer reviewed publication published as of Apr. 20, 2022. There are numerous peer reviewed publications reporting on numerous therapeutic agents (aka drugs) that have demonstrated neuroplasticity effects such as side benefits on the neurorestorative physiologic processes including neurogenesis. Specifically numerous peer reviewed publications report an extensive variety of drugs (150+ as of the filing of this application) that influence the neurorestorative processes as a side effect of numerous existing molecules. Admittedly, little, if any, follow-up clinical research has been done to evaluate the capacity of these drugs to promote neurorestoration or to better understand the capacity of any one molecule to preferentially effect or attenuate the function of any of the key neurorestorative physiologic processes instrumental in the creation of new neurons, axons, or dendrites, or Remyelination. Interestingly, a majority of these molecules or agents with proven neuroplasticity effects are already commercial, off-patent, and FDA approved for other indications, i.e., safe for human use. The phrase Approved by an appropriate regulatory agency for medical use in human patients means approved by the FDA for any indication for human use by Apr. 20, 2022. This Approved status yields a collection of clinical data for each agent.

Thus the combinatorial pharmacological compositions 100 of the invention are engineered using agents 30, 40 or 50 already approved by the FDA for other indications. There are over 150 FDA-approved agents with well reported neuroplastic properties. The benefits of repurposing agents are: i) the cost of reformulating and repurposing agents 30, 40 and 50 can be <20% of the cost associated with the production of a brand-new drug, ii) the time to market and to treating patients can be <⅓^{rd} of that for a brand-new drug; iii) the risk of adverse interactions and regulatory agency denial is far less as the toxicity and chemical/biological properties of the repurposed agents 30, 40 and 50 are well known and large patient data sets exist. Using existing, safe agents 30, 40 and/or 50 the present framework engineers and combines multiple agents 30, 40 and 50 that have different mechanisms of plasticity (discussed below) so to target the dynamic and complex nature of the brain and brain ailments.

The already commercial molecules or agent (30, 40 or 50) allow for a significantly more cost-effective regulatory approval/commercialization pathway in many jurisdictions. This pathway allows for accelerated, lower cost approvals, as long as the already approved, i.e., determined safe for human use, molecule or agent 30, 40 or 50 is not altered. As detailed below the combinatorial pharmacological compositions 100 may change dosing (to a lower amount then previously approved), delivery methods (oral, transdermal, injectable, etc.), and certainly the indication, but not the structure of molecule or agent. Many of the combinatorial pharmacological compositions 100 according to the invention will more than two such molecules or agents 30, 40 or 50. Being able to bring a life changing neurorestorative therapy to the market quickly with the combinatorial pharmacological compositions 100 of the invention will result in significant benefits to many sufferers of neurologic deficits, e.g., stroke.

The present invention utilizes a pharmacologically effective amount of Neuroplasticity PA therapeutic agents 30, 40 or 50 (or more). The an pharmacologically effective amounts are amounts up to the approved dosages of the Neuroplasticity PA therapeutic agents for other indications, as these have been deemed to be safe and effective dosages. The combinatorial pharmacological compositions 100 utilizes multiple agents 30 40 and/or 50 and these may have synergistic effects such that the pharmacologically effective amount of any agent 30, 40 or 50 may be less than the previously regulatory agency approved dosages for the agents when administered individually. Each combinatorial pharmacological composition 100 of the present invention may include a vasodilator or other penetrating/altering agent to increase flow across the blood brain barrier, and this aspect may further decrease the pharmacologically effective amount of any agent 30, 40 or 50. The ability to effectively utilize less than the previously regulatory agency approved dosages for the agents in the present invention is referenced as micro-dosing of the agents 30, 40 and 50.

### Vasodilator

As noted above another aspect of this invention combines the use of one or more molecules or agents 30, 40 or 50 in the combinatorial pharmacological composition 100 in conjunction with a vasodilator. A vasodilator opens the arteries and increases the flow of blood to the brain. Concurrently, this increased blood flow increases the volume of any and all molecules in the blood including, but not limited to: nutrients, stem cells, Growth Factors, (e.g., VEGF), oxygen, and any neurorestorative molecules that promote plasticity disclosed herein. It has been suggested that the physical expansion of the blood vessels also expands the BBB openings. There are many known methods of enhanced BBB crossing that may be implemented including those discussed in Furtado D, Björnmalm M, Ayton S, Bush A I, Kempe K, Caruso F. Overcoming the Blood-Brain Barrier: The Role of Nanomaterials in Treating Neurological Diseases. Adv Mater. 2018 November; 30(46):e1801362. doi: 10.1002/adma.201801362. Epub 2018 Jul. 31. PMID: 30066406.

### Matrix of Mechanism of Action 10

This present invention focuses on the restoration of neuro-function damaged or lost as a result of disease or trauma. The present invention provides a combinatorial pharmacological composition which combines two or more of the 150+ molecules or agents 30, 40 or 50 known to enhance neuro-restoration (i.e., the creation of neurons, axons, synapses, and or remyelination to restore brain function) and implemented within a combinatorial pharmacotherapy, each of the molecules or agents 30, 40 or 50 of the combinatorial pharmacological composition 100 targets a different combination of the mechanism of action 10 or each of which effects a mechanism of action 10 differently. The mechanism of action 10 may be defined as detailed below as a matrix forming relevant or key neurorestorative physiologic processes for evaluating the agents 30 40 or 50 forming the combinatorial pharmacological composition 100.

There is accepted knowledge regarding various neurorestorative physiologic processes (selective ones of which form the matrix defining the mechanisms of action 10) with the human brain that either promote or suppress neuro-restoration. The present invention targets the attenuation of neurorestorative physiologic processes individually and concurrently. The present invention categorizes a number of neurorestorative physiologic processes some of which are inhibitory (they are to be reduced) and others that are excitatory (they are to be augmented): Those that are inhibitory or to be reduced include Glial Scar; Apoptosis 24; and Neuro-inflammation or Inflammation 22. Those that are Excitatory or to be augmented include: Neurogenesis 12; Bioenergetics 18 (AKA Mitochondrial protection; Remyelination 16; Angiogenesis 20; and synaptogenesis 14. The present invention defines seven of these neurorestorative physiologic processes as the seven key components or matrix forming mechanism of action 10 for evaluating the agents 30 40 or 50 forming the combinatorial pharmacological composition 100.

Glial scar formation (also known as gliosis) is a reactive cellular process involving astrogliosis that occurs after injury to the central nervous system. As with scarring in other organs and tissues, the glial scar is the body's mechanism to protect and begin the healing process in the nervous system. In the context of neurodegeneration, formation of the glial scar has been shown to have both beneficial and detrimental effects. Particularly, many neuro-developmental inhibitor molecules are secreted by the cells within the scar that prevent complete physical and functional recovery of the central nervous system after injury or disease. In an alternative embodiment Glial scar formation may be added to the matrix forming the mechanism of action 10.

Apoptosis 24 is a form of programmed cell death that occurs in multicellular organisms. Biochemical events lead to characteristic cell changes (morphology) and death. These changes most generally include blebbing, cell shrinkage, nuclear fragmentation, chromatin condensation, DNA fragmentation, and mRNA decay. For reference, the average adult human loses between 50 and 70 billion cells each day due to apoptosis. For an average human child between eight and fourteen years old, approximately twenty to thirty billion cells die per day. In contrast to necrosis, which is a form of traumatic cell death that results from acute cellular injury, apoptosis is a highly regulated and controlled process that confers advantages during an organism's life cycle. Unlike necrosis, apoptosis produces cell fragments called apoptotic bodies that phagocytes are able to engulf and remove before the contents of the cell can spill out onto surrounding cells and cause damage to them. Apoptosis can be initiated through one of two pathways. In the intrinsic pathway the cell kills itself because it senses cell stress, while in the extrinsic pathway the cell kills itself because of signals from other cells. Weak external signals may also activate the intrinsic pathway of apoptosis. Both pathways induce cell death by activating caspases, which are proteases, or enzymes that degrade proteins. The two pathways both activate initiator caspases, which then activate executioner caspases, which then kill the cell by degrading proteins indiscriminately. In addition to its importance as a biological phenomenon, defective apoptotic processes have been implicated in a wide variety of diseases. Excessive apoptosis causes atrophy, whereas an insufficient amount results in uncontrolled cell proliferation, such as cancer. Some factors like Fas receptors and caspases promote apoptosis, while some members of the Bcl-2 family of proteins inhibit apoptosis.

Neuroinflammation or simply inflammation 22 is inflammation of the nervous tissue. It may be initiated in response to a variety of cues, including infection, traumatic brain injury, toxic metabolites, or autoimmunity. In the central nervous system (CNS), including the brain and spinal cord, microglia are the resident innate immune cells that are activated in response to these cues. The CNS was typically considered an immunologically privileged site because peripheral immune cells are generally blocked by the blood brain barrier (BBB), a specialized structure composed of astrocytes and endothelial cells. However, following injury to the CNS circulating peripheral immune cells may surpass a compromised BBB and encounter neurons and glial cells expressing major histocompatibility complex molecules, perpetuating the immune response. Brain-resident cells, including microglia and astrocytes also generate an inflammatory response following various forms of brain injury. Although the response is initiated to protect the central nervous system from the infectious agent, the effect may be toxic and widespread inflammation as well as further migration of leukocytes through the blood brain barrier. Neuroinflammation is implicated in contributing to a variety of neurologic and somatic illnesses including Alzheimer's disease (AD), Parkinson's disease (PD), and depression.

Neurogenesis 12 is the process by which nervous system cells, the neurons, are produced by neural stem cells (NSCs). It occurs in all species of animals (except the porifera (sponges) and placozoans). Types of NSCs include neuroepithelial cells (NECs), radial glial cells (RGCs), basal progenitors (BPs), intermediate neuronal precursors (INPs), subventricular zone astrocytes, and subgranular zone radial astrocytes, among others. Neurogenesis is most active during embryonic development and is responsible for producing all the various types of neurons of the organism, but it continues throughout adult life in a variety of organisms. Once born, neurons do not divide (e.g., mitosis), and many will live the lifespan of the animal. Neurogenesis has also been described as the formation of new neurons from neural stem and progenitor cells which occurs in various brain regions such as the subgranular zone of dentate gyrus in the hippocampus and the subventricular zone of lateral ventricles.

Bioenergetics 18 relates to the improvement of mitochondrial function, and includes mitochondrial protection. Within the brain, mitochondria serve as the primary producers of ATP to meet the high energy requirements of individual neurons. Through their electron transport chains (ETC), mitochondria generate most of this ATP in an oxygen-dependent manner. with toxic reactive oxidative stress (ROS) also released as a by-product of the same process. Over time an accumulation of this ROS can severely damage all of the cell types in the brain, ultimately causing death of the affected cells. Mitochondrial dysfunction is often implicated in disorders of the brain, in particular Parkinson's disease (PD), an incurable movement disorder caused by the progressive neurodegeneration of dopaminergic neurons (DA). Compared to other neurons, DA neurons are more vulnerable to ROS due to their intrinsic pacemaking ability. As a consequence. these neurons are under constant oxidative stress that can cause irreparable damage to mitochondria. Bioenergetics 18 broadly defines the improvement of mitochondrial function. Mitochondrial protection is literally the action or mechanism of protecting the mitochondria.

Remyelination 16 is the process of propagating oligodendrocyte precursor cells to form oligodendrocytes to create new myelin sheaths on demyelinated axons in the central nervous system. This is a process naturally regulated in the body and tends to be very efficient in a healthy central nervous system. The process creates a thinner myelin sheath than normal, but it helps to protect the axon from further damage, from overall degeneration, and proves to increase conductance once again. The processes underlying Remyelination 16 are under investigation in the hope of finding treatments for demyelinating diseases, such as multiple sclerosis.

Angiogenesis 20 is the physiological process through which new blood vessels form from pre-existing vessels, formed in the earlier stage of vasculogenesis. Angiogenesis 16 continues the growth of the vasculature by processes of sprouting and splitting. Vasculogenesis is the embryonic formation of endothelial cells from mesoderm cell precursors, and from neovascularization, although discussions are not always precise (especially in older texts). The first vessels in the developing embryo form through vasculogenesis, after which angiogenesis 20 is responsible for most, if not all, blood vessel growth during development and in disease. Angiogenesis 20 is a normal and vital process in growth and development, as well as in wound healing and in the formation of granulation tissue. However, it is also a fundamental step in the transition of tumors from a benign state to a malignant one, leading to the use of angiogenesis inhibitors in the treatment of cancer.

Synaptogenesis 14 is the physiological process increasing neuron connectivity. Specifically synaptogenesis is the formation of synapses between neurons in the nervous system. Although it occurs throughout a healthy person's lifespan, an explosion of synapse formation occurs during early brain development, known as exuberant synaptogenesis.

The present invention focuses on the new treatment modality of synergistic combination pharmacotherapy applied to neurorestoration which seeks to balance and optimize the aforementioned neurorestorative physiologic processes of the mechanism of action 10 matrix to counter current pathophysiology or deficits accrued through disease or trauma. One aspect of the present invention provides a method for designing a combinatorial pharmacological composition 100 and associated treatment protocol that enhances neurorestoration based upon select agents 30, 40 or 50 capacity to attenuate one or more of the seven mechanisms of action causing or associated with the neural deficit. Each of two or more selected molecules or agents 30, 40 or 50 will have a property to excite, inhibit or attenuate one of the following:

Matrix 10 of the present invention formed by 7 key neurorestorative physiologic processes forming the Mechanisms of Action: Inhibitory: 1) Apoptosis 24 and 2) Neuro-inflammation 22; Excitatory: 3) Synaptogenesis 14, 4) Neurogenesis 12, 5) Bioenergetics 18 6) Remyelination 16 and 7) Angiogenesis 20.

### Mechanism of Plasticity 32, 42 and 52

The matrix of key neurorestorative physiologic processes define the matrix or mechanism of action 10 of the present invention. Those neurorestorative physiologic processes that are positively effective by any Neuroplasticity PA therapeutic agent 30, 40 or 50 define the mechanism of plasticity 32, 42 or 52 for that agent 320, 40 or 50. The primary and secondary (and other) neurorestorative physiologic processes for Neuroplasticity PA therapeutic agents 30, 40 or 50 are known, although most are understudied. Influence on these processes is demonstrated by animal tissue and cellular models in neurologic disease states. The mechanism of plasticity 32, 42 or 52 for a specific Neuroplasticity PA therapeutic agent 30, 40 or 50 can be viewed as mapping the Neuroplasticity PA therapeutic agents 30, 40 or 50 onto the matrix of mechanism of action 10.

The matrix 10 developed herein enables the optimal development and selection of Neuroplasticity PA therapeutic agents 30, 40 or 50 combinations to enhance neurorestoration. The utility of the matrix 10 will be improved by the integration of AI and or ML algorithms for the design of therapeutic combinations and further study. Another aspect of this invention implements the use of two or more agents in combination that target one or more of the identified neurorestorative physiologic processes as discussed below.

The Neuroplasticity PA therapeutic agent 30, 40 or 50 combinations developed here create an optimal environment within which neurorestoration can happen, allowing individual neurons, synapses, and whole neural networks to experience enhanced and efficient reconfiguration. Rehabilitation should ideally take advantage of this plasticity by exercising, stimulating, and enhancing configurations that are beneficial and therapeutic to the patient. Because of this, targeted stimuli and rehabilitative tasks that exercise valuable functions as well as associated axonal tracts are a logical choice.

As noted above the present invention provides a combinatorial pharmacological composition 100 which combines two or more of the (120+) Neuroplasticity PA therapeutic agents (30, 40 or 50) known to enhance neuro-restoration (i.e., the creation of neurons, axons, synapses, and or myelin sheaths to restore brain function) and implemented within a combinatorial pharmacotherapy. The combinatorial pharmacotherapy developed under this invention may further be in combination with a structured, clinically supervised rehabilitation protocol (for example but not limited to: physical therapy, VR/AR Gaming therapy, exercise, or repetitive daily activity). Necessary daily activities can provide sufficient repetition to promote the rebuilding of the necessary neuronal, axonal, synaptic, and or myelin pathways to restore/enhance function in the presence of an enhanced neurologic environment for the creation of new neurons, axons, synapsis, and or myelin sheaths.

One method in accordance with the present invention may be described as following general principles of operation: In the presence of a neurological dysfunction, disorder or desired neurological outcome (see Disorders and Conditions list, below), apply the combinatorial molecule therapeutic intervention formed by the combinatorial pharmacological composition 100 to increase neuroplasticity. During application of the pharmacological therapy, perform simultaneous repetitive tasks as rehabilitation for the dysfunction or disorder at the clinician's discretion. Periodically (on the scale of hours, days, weeks, or according to clinical schedule) assess the success of therapeutic intervention by applying a specific set of tests that assess the neuro-motor, sensory, cognitive or other neurological performance targeted. Assessment may also be performed immediately or in real-time during task performance. During the task, immediate visual feedback may be provided to the patient to further enhance learning and provide real-time immediate assessment. This feedback may be customized with feedback target goals. Neurological rehabilitation of subjects with pharmacologically induced neuroplasticity using oculomotor visual tasks with a video-occulography (VOG) system is disclosed in the above mentioned WO 2020/097320 which is incorporated herein by reference. Another manifestation of the method will be a combination that includes physical exercise and or physiological stress regimen that is specifically intended to improve neuroplasticity in a neurodegenerative or brain injury context, e.g. in stroke, spinal cord injury or traumatic brain injury (TBI) recovery. Research has established that repetition builds pathways . . . certain types of exercise regimens, in part through increased BDNF.

In the various tasks, the participant engages in various cognitive, sensory, neuro-motor other therapeutic neurological activities. In the presence of a neuroplasticity enhancing therapy the participants are instructed to execute a task(s) aligned with the functions and as directed by a clinician. The means of evaluating the effect of this combinatorial therapy include but are not restricted to objective biomarkers such as neuroimaging (MRI, fMRI, PET, intracranial blood flow), serum biomarkers, oculomotor testing, neuropsychiatric and cognitive testing, neuromuscular and sensory performance etc.

### Disorders, Conditions and Desired Effects

The following is a list of disorders and conditions which may benefit from the proposed method, and for which rehabilitation is appropriate and feasible. This list provides examples, and is not meant to be an exhaustive enumeration of all conditions to which the proposed method can be applied. The pharmacotherapeutic protocol according to the present invention may provide specifically for patient who is suffering from i) an injury to the central nervous system comprising at least one of stroke, TBI, PTSD, CTE and Spinal Cord Injury; ii) from at least one of dementia and movement disorders comprising at least one of Alzheimer's disease, Parkinson's disease and ALS.; iii) from at least one of headaches and depression comprising at least one of migraine, depression and Bipolar disorder; iv) from an immune system attack comprising at least one of MS, Muscular Dystrophy, Rheumatoid Arthritis and Lupus; v) from a neurological or psychological disorder comprising at least one of Cerebral Palsy, Schizophrenia, Epilepsy, Autism, and Dyslexia; vi) from a cognitive deficit comprising at least one of long COVID, Cancer Chemo Fog, Anesthetic fog, Chronic fatigue and Fibromyalgia; vii) deficit associated with prosthetic limb operation and/or Limb rehabilitation/enhancement; viii) deficit associated with Brain injury due to exposure to neurotoxins, and for pain management; and ix) deficits in or need for learning enhancement such as language or mathematics.

### Development of Combinatorial Pharmacological Composition 100 for Stroke Patients

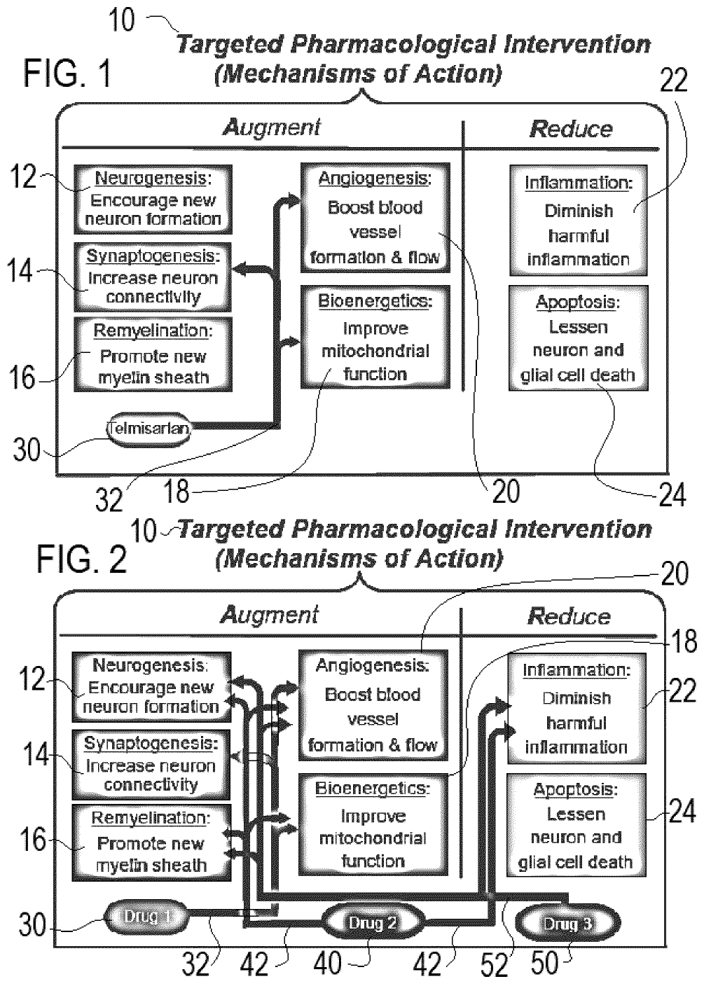

FIGS. 1-3 disclose the development of a combinatorial pharmacological composition 100 and associated protocol engineered for patients suffering from chronic motor deficits due to stroke. There is a particular need for the combinatorial pharmacological composition 100 and associated protocol in this patient population because: this population represents a large, growing, unmet medical need. Stroke patients are a 7 million patient cohort in the US and 100 million globally. One in four adults are expected to suffer a stroke in their lifetime Furthermore this population has Objective endpoints to effectively validate clinical efficacy (patient gains). There are well-established objective clinical outcome measures to show improvements in patients' motor skills. Using these validation measures increases the likelihood of FDA approvals.

Recent clinical studies have proven that intensive exercise and stimulation programs can restore some lost function to stroke patients, even years after a stroke. Unfortunately, most patients are still left with deficits in motion, speech, cognition, and other critical functions. The combinatorial pharmacological composition 100 and associated protocol outlined in FIGS. 1-3, is designed to increase brain neuroplasticity, so that patients can get more motor function recovery. Rehabilitation therapies in general vary widely and do not produce consistent beneficial gains in patient functionality. Adding the pharmacological component 100 to a rehabilitation protocol will meaningfully improve patient outcomes. Based on the 100-point FUGL Meyer Scale, gold standard measure of stroke function, the combinatorial pharmacological composition 100 and associated protocol will add at least 3 points to this score, translating into functional patient gains that improve patient quality of life.

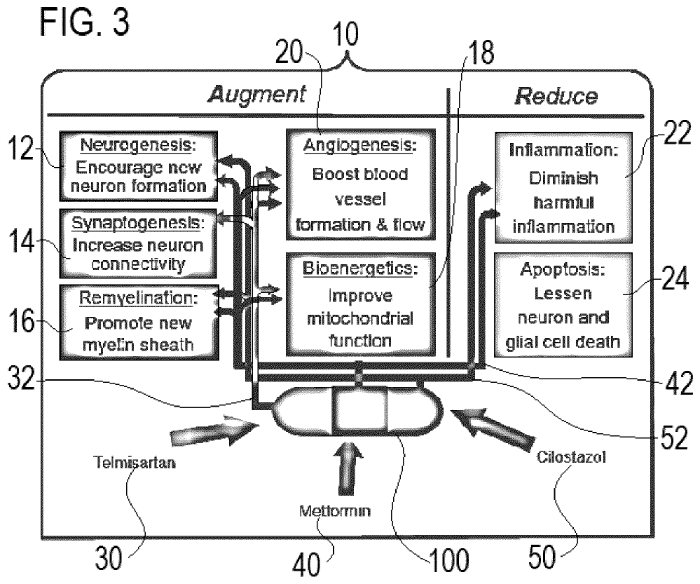

This embodiment of the invention may be described as a pharmacotherapeutic protocol enhancing neuro-restoration in the human brain for patients suffering with stroke, wherein the protocol comprises treating the patient with a combinatorial pharmacological composition 10 comprising a pharmacologically effective amount of a first Neuroplasticity PA therapeutic agent 30 Telismartin exhibiting a mechanism of plasticity 32 in Synaptogenesis, Angiogenesis, and Bioenergetics, and a pharmacologically effective amount of a second Neuroplasticity PA therapeutic agent 40 metformin exhibiting a mechanism of plasticity 42 in at least Neurogenesis, Remyelination, Angiogenesis, Bioenergetics, and Inflammation and a pharmacologically effective amount of a third Neuroplasticity PA therapeutic agent 50 Cilostazol exhibiting a mechanism of plasticity 52 in Neurogenesis, Remyelination, Angiogenesis, and Inflammation. FIG. 1 schematically illustrates the mechanism of plasticity 32 of a Neuroplasticity PA therapeutic agent 30 Telismartin forming a combinatorial pharmacological composition 100 enhancing neuro-restoration in the human brain for patients suffering with stroke according to one representative example of the present invention; FIG. 2 schematically illustrates the mechanisms of plasticity 32, 42 and 52 of three Neuroplasticity PA therapeutic agents 30, 40 and 50 forming the combinatorial pharmacological composition 100 for stroke; and FIG. 3 schematically illustrates the mechanism of plasticity of the combinatorial pharmacological composition 100 for stroke comprising the three agents 30, 40 and 50.

TELMISARTAN—Telmisartan has a chemical formula C₃₃H₃₀N₄O₂ is an angiotensin II receptor blocker that shows high affinity for the angiotensin II receptor type 1 (AT₁), with a binding affinity 3000 times greater for AT₁ than AT₂. In addition to blocking the renin-angiotensin system, Telmisartan acts as a selective modulator of peroxisome proliferator-activated receptor gamma (PPAR-γ), a central regulator of insulin and glucose metabolism. Telmisartan's dual mode of action may provide protective benefits against the vascular and renal damage caused by diabetes and cardiovascular disease (CVD). Telmisartan demonstrates activity at the peroxisome proliferator-activated receptor delta (PPAR-δ) receptor and activates PPAR-δ receptors in several tissues. Also, Telmisartan has a PPAR-γ agonist activity. Effective amounts of Telmisartan in the single oral dose (e.g. pill) of the compound of the present invention is less than 80 Mg, more preferably less than 40 Mg, and most preferably less than 20 Mg per dose.

METFORMIN—Metformin has a formula CaHnNs and is well established as a main first-line medication for the treatment of type 2 diabetes, particularly in people who are overweight. It is also used in the treatment of polycystic ovary syndrome. Metformin is generally regarded as safe and well-tolerated. Metformin is a biguanide drug that reduces blood glucose levels by decreasing glucose production in the liver, decreasing intestinal absorption, and increasing insulin sensitivity. Metformin decreases both basal and postprandial blood glucose levels. In PCOS, Metformin decreases insulin levels, which then decreases luteinizing hormone and androgen levels. Effective amounts of Metformin in the single dose of the compound of the present invention is less than 850 Mg, more preferably less than 500 Mg, and most preferably less than 250 Mg per dose. Metformin may be less than 125 Mg per dose of the compound of the present invention.

CILOSTAZOL—Cilostazol has a formula C₂₀H₂₇N₅O₂ and is a selective inhibitor of phosphodiesterase, which in turn increases the activation of intracellular cAMP and thereby inhibits platelet aggregation. An increase in cAMP results in an increase in the active form of protein kinase A (PKA), which is directly related with an inhibition in platelet aggregation. PKA also prevents the activation of an enzyme (myosin light-chain kinase) that is important in the contraction of smooth muscle cells, thereby exerting its vasodilatory effect. Cilostazol has been noted as a powerful alternative to aspirin in certain aspects. In previous clinical trials for example, cilostazol has been found to significantly reduce the incidence of recurrent stroke, with fewer hemorrhagic events, compared with aspirin. Sec Huang Y, Cheng Y, Wu J, Li Y, Xu E, Hong Z, et al; Cilostazol versus Aspirin for Secondary Ischaemic Stroke Prevention Cooperation Investigators. Cilostazol as an alternative to aspirin after ischaemic stroke: a randomised, double-blind, pilot study. Lancet Neurol. 2008; 7:494-499. See also Nakamura T, Tsuruta S, Uchiyama S. Cilostazol combined with aspirin prevents early neurological deterioration in patients with acute ischemic stroke: a pilot study. J Neurol Sci. 2012; 313:22-26. Effective amounts of Cilostazol in the single oral dose of the compound of the present invention is less than 200 Mg, more preferably less than 100 Mg, and most preferably less than 50 Mg. The effective amount of Cilostazol in the compound of the present invention may be less than 25 Mg.

With the development of any new therapeutic it is critical to ensure an appropriate safety profile, such that the potential benefits exceeds any reasonably anticipated risks. Safety considerations are advantageous in reviewing the three drugs or agents 30, 40 and 50 (Telmisartan Metformin and Cilostazol) that make up combinatorial pharmacological composition 100 for stroke rehabilitation. With respect to safety, the combinatorial pharmacological composition 100 for stroke may be considered regarding several distinct features of the drugs or agents 30, 40 and 50 alone and in combination; the well-known and appreciated common adverse effects based on the extensive use of each drug or agent 30, 40 and 50, as well as any possible known pharmacokinetic interaction when the drugs 30, 40 and 50 are combined, and any specific considerations related to the use of the combinatorial pharmacological composition 100 for stroke in an older population with cardiovascular disease.

The chosen drugs or agents 30, 40 and 50 (Telmisartan Metformin and Cilostazol) in the combinatorial pharmacological composition 100 for stroke have each individually been in widespread use for more than 20 years. With this experience the more frequent adverse events are well-recognized and reported in detail on drug information web sites such as Drugs.com. These information sources establish the combinatorial pharmacological composition 100 for stroke avoided drugs that had significant risk profiles or Black Box Warnings. The chosen drugs do, of course, still have known adverse effects. For example, metformin often results in gastrointestinal upset, while cilostazol is known to trigger headaches in some patients. Importantly, the known adverse effects are different for each agent, so that the risk that would arise if all of the drugs, for example, caused headache can be avoided.

The combinatorial pharmacological composition 100 for stroke implementing the chosen drugs or agents 30, 40 and 50 (Telmisartan Metformin and Cilostazol) avoids pharmacokinetic (PK) interactions. The main routes of drug elimination from the body include filtration by the kidney, metabolism by cytochrome P450 enzymes in the liver, and/or by conjugation and elimination by the liver in bile. A well-known source of drug-drug interactions (DDIs) is the interference with the metabolism of one drug by a second that inhibits or induces a cytochrome P450 enzyme. If this interaction occurs it can result in unexpectedly high drug concentrations in blood which carries the risk of triggering adverse effects. However, the interaction of drugs with cytochrome P450 enzymes is carefully evaluated and reported during development, and is thus well-understood. The combinatorial pharmacological composition 100 for stroke implementing the chosen drugs or agents 30, 40 and 50 (Telmisartan Metformin and Cilostazol) with distinct routes of metabolism and excretion avoiding interactions that would change drug exposures when combined. Metformin is excreted unchanged by the kidney and liver. Cilostazol is a substrate for CYP3A4, but the other drugs do not interact with CYP3A4. Telmisartan in conjugated in the liver and excreted without further metabolism into the bile. Thus, the drugs have separate and independent mechanisms of excretion and are thus very unlikely to result in any PK interactions or DDIs.

There are special considerations for patients who have suffered a stroke. The factors that contribute to stroke risk are complex, but include age, hypertension, diabetes, and other cardiovascular disease amongst others. In treating individuals after they have experienced a stroke it is recognized that such individuals are also at risk of a subsequent stroke. As such, it is important to not increase the risk of stroke and the combinatorial pharmacological composition 100 for stroke implementing the chosen drugs or agents 30, 40 and 50 (Telmisartan Metformin and Cilostazol) is beneficial in this regard. Cilostazol, as an antiplatelet drug is actually used for stroke prevention, so it does not increase the risk of stroke. Telmisartan can be used for the management of hypertension, which may also reduce the risk of stroke. Metformin is widely used to treat type 2 diabetes which, if untreated, also increases stroke risk. Thus, our drug combination clearly does not present additional risks to the post-stroke population that we propose to treat.

### Therapeutic Intervention List

The present invention utilizes Neuroplasticity PA therapeutic agents as defined above that are generally known. The following discussion lists potential components for the composition 100, most of which are Neuroplasticity PA therapeutic agents. Those of the following listing that are Neuroplasticity PA therapeutic agents (again meaning they form a therapeutic agent that has Proven neuroplasticity effects and has been Approved by an appropriate regulatory agency for medical use in human patients.) they are implemented as discussed above implementing the matrix 10 and the agents mechanism of plasticity to define complementary combinations. Components below that are not Neuroplasticity PA therapeutic agents because they lack the approval can also follow the use of the matrix. Components below that are not Neuroplasticity PA therapeutic agents because they lack the proven effects would be generally not utilize the matrix 10 in investigating their use of addition.

For example, one manifestation of the invention will be the combination of fluoxetine to stimulate angiogenesis with memantine to suppress neuro-inflammation and candesartan to dilate the blood vessels. Particularly these medications have been shown to increase plasticity in hippocampal dentate gyrus (DG) cells, which is a well-documented site underlying new learning. Further research has demonstrated increases in neuroplasticity or markers of neuroplasticity in other areas such as the visual cortex, amygdala, and medial pre-frontal cortex. In patients with mood disorders, SSRIs have been shown to be most effective when combined with other therapy, e.g., Cognitive-Behavioral, CBT, supporting the potential benefit of combining neuroplasticity (from SSRIs) with a targeting intervention (CBT). The method described in this invention employs the same physiological principles, but using an intervention that exercises more fundamental neuro-behavioral systems.

Selective Serotonin Reuptake Inhibitors (SSRIs)—This list is meant to be demonstrative and not exhaustive in term of use with the process outlined herein. The following is a list of SSRIs suitable for the methodology of the present invention: Citalopram, Escitalopram, Fluoxetine, Fluvoxamine, Paroxetine, Sertraline, and Vilazodone.

Brain Derived Neuro-Trophic Factor (Bdnf) Action In Promoting Neuroplasticity-Another set of selective agents for selection in the present invention or those agents that includes pharmacological therapies designed to enhance Brain Derived Neuro-trophic factor (BDNF) action in promoting neuroplasticity. BDNF signaling, particularly through its TrkB receptor target, forms a critical component in multiple types of neuroplasticity-enhancing interventions. Evidence suggests that its expression is influenced by increased neural activity, which rehabilitation tasks are meant to provide. BDNF enhancing therapeutics include, but are not limited to: ketamine and its metabolic derivatives norketamine and hydroxynorketamine (HNK); memantine; riluzole; Quercetin; Therapeutic administration of botanicals with BDNF effect, e.g., ginsenosides, salidroside, glycosides, Ginkgo biloba, Hypericum perforatum; Artesunate and Clemastine.

Steroids—Another manifestation of the invention will be a combination that includes steroids, such as: Neurosteroids (Pregnenolone, Dehydroepiandrosterone, Allopregnanolone, and their synthetic analogs). Neurosteroids can affect neuroplasticity and neurogenesis through their actions on DNA gene transcription and possibly more directly through neurotransmitter receptors and receptor modulation; Sex steroids, i.e. testosterone, estrogen, and progesterone. These steroids have strong effects on general neuroplasticity, and this manifestation of the method incorporates their potential benefit in rehabilitative therapy.

Pharmacological Psychedelics—Another manifestation of the invention is a combination that includes pharmacological psychedelics, which have been shown to promote neuroplasticity both structurally and functionally, including but not limited to: tryptamines (N,N-dimethyltryptamine [DMT] and psilocin); amphetamines (2,5-dimethoxy-4-iodoamphetamine [DOI] and MDMA); and ergolines (lysergic acid diethylamide [LSD]).

Other Therapeutic Agents—Another manifestation of the invention will be a combination that includes other therapeutic agents and methods not mentioned above, that induce neuroplasticity and neurogenesis, including but not limited to: Stem cells, Exosomes and other cellular therapies; Valproic Acid; Non-SSRI antidepressants; NDRI's, lithium carbonate, heterocyclic antidepressants, Metformin, N-Acetylcystine, Human Growth Hormone

Additional Agents—The following represents additional substances that can be used to moderate the functionality of one or more of the 7 mechanisms of action instrumental in managing neurogenesis: Selective Norepinephrine and Serotonin Reuptake Inhibitors (SNRIs) Desvenlafaxine, Duloxetine, Levomilnacipran, Milnacipran, Venlafaxine; Tricyclic and Heterocyclic Antidepressants including Amitriptyline, Amoxapine, Desipramine, Doxepin, Imipramine, Nortriptyline, Protriptyline, Trimipramine, Trazodone, and Maprotiline; Dopamine and mixed Dopamine and Serotonin Reuptake Inhibitors including Bupropion, Amineptine, Ethylphenidate, Modafinil, Armodafinil, Vanoxerine, Amantadine, Benztropine, Methylphenidate, Rimcazole; Tetracyclic Antidepressants including Mirtazipine and Maprotiline; Monoamine Oxidase Inhibitors—Isocarboxazid, Phenelzine, Rasagiline, Selegiline, Tranylcypromine and Methylene Blue; Anticonvulsants including Valproic Acid and Ethosuximide; Plant and Fungi Derived Substances including Curcumin, Aristoforin, Quercetin, Artesunate, N-Acetylcysteine, Lion's Mane Mushroom extract, Psilocin, Mescaline, LSD, DOI, DMT; Muscarinics including Galantamine, Rivastigmine, Memantine, and Donepezil; Endocannabinoid System such as Cannabidiol; NMDA Receptor Antagonist family such as NMDA, Ketamine, Esketamine, Norketamine, HNK, NV-5138 Leucine, Apimostinel, and Rapastinel; Immunomodulators such as NeuroStat, Cyclosporine A, Dexamethasone, Naltrexone, Naloxone, Prednisolone, Methylprednisolone, Fluticasone; Hormone Based Medications such as Progesterone, Estrogens, Flavinoids, Resveratrol, Human Growth Hormone (various isomers), Ghrelin and Melatonin; Anti-Inflammatory Drugs such as Antithrombin, Celecoxib, Dexamethasone and Various steroid drugs; Miscellaneous Drugs such as Lithium and Minocycline; and Other Biological Agents such as Anti Nogo antibodies, Bacterial Enzyme Chondroitinase ABC (ChABC), Bacterial Toxin VX-210, Metalloproteinases, MMP 2 and 9 antibodies and inhibitors

### ADDITONAL REPRESENTATIVE EXAMPLES

### 1.) Pentoxifylline Plus N-Acetylcysteine Plus Lithium Carbonate

Vasodilator—Pentoxifylline. Pentoxifylline (PTX) is a phosphodiesterase inhibitor with potent anti-inflammatory and antioxidant effects, with additional pleiotropic effects that lead to improved CBF and increases in brain derived neurotrophic factor (BDNF) levels. It is also an ancient drug with a good safety profile and has been off patent for a long time. N-acetylcysteine (NAC) is a glutathione precursor with potent antioxidant, pro-neurogenesis and anti-inflammatory properties and a favorable safety profile. Most studies of NAC have focused on neurons. However, neuroprotection may be complemented by the protection of astrocytes because healthier astrocytes can better support the viability of neurons. NAC can protect astrocytes against protein misfolding stress. Lithium-mediated neuroprotective and neurogenic effects involve mechanisms highly relevant to the post-stroke population including the increased expression of brain-derived neurotrophic factor (BDNF) and Bcl-2, and inhibition of GSK-3β. In vivo studies also reveal that lithium-treated rats compared to controls had a significant increase in MANF (a form of BDNF) expression in the Prefrontal Cortex and Striatum.

### 2.) Sildenafil Plus Galantamine Plus Escitalopram

Vasodilator—Sildenafil: Sildenafil produces vasodilation of vascular beds in the brain, lungs, heart and penis by increasing the stability of cyclic guanosine 3′-5′-monophosphate (cGMP). Increased stability of cGMP effectively raises the intracellular level of cGMP. Elevated cGMP relaxes the smooth muscle of blood vessels via the Nitric Oxide pathway which allows increased blood flow to the cerebral and meningeal arteries. This effect augments the delivery of therapeutic agents and endogenous growth factors to injured areas of the brain. Sildenafil also beneficially affects the following: Control of Neuro-inflammation and Angiogenesis. Galantamine is a reversible, competitive acetylcholinesterase inhibitor, used to treat patients with Alzheimers disease. It has been demonstrated that galantamine increases cerebral neurogenesis and has a neuroprotective effect by binding to nicotinic receptors and has an anti-inflammatory effect due to its allosteric binding to the α7nAChR. Escitalopram is an SSRI medication that increases neurogenesis via a Non BDNF pathway. It can also decrease oxidative stress and potentiation of neurite outgrowth.

### 3.) Sildenafil Plus Valproic Acid Plus Melatonin

Vasodilator—Sildenafil. Sildenafil produces vasodilation of vascular beds in the brain, lungs, heart and penis by increasing the stability of cyclic guanosine 3′-5′-monophosphate (cGMP). Increased stability of cGMP effectively raises the intracellular level of cGMP. Elevated cGMP relaxes the smooth muscle of blood vessels via the Nitric Oxide pathway which allows increased blood flow to the cerebral and meningeal arteries. This effect augments the delivery of therapeutic agents and endogenous growth factors to injured areas of the brain. Sildenafil also beneficially affects the following: Control of Neuro-inflammation and Angiogenesis Valproic Acid mechanism of actions involves histone deacetylase inhibition and upregulation of hypoxia-inducible factor-1α and its downstream proangiogenic factors vascular endothelial growth factor and matrix metalloproteinase-2/9. Chronic VPA treatment enhances angiogenesis and promotes functional recovery after brain ischemia. Apart from promoting neurogenesis, melatonin enhances survival and dendritic maturation of the immature neurons in the hippocampus. Melatonin promotes viability, proliferation, and neuronal differentiation of mouse embryonic cortical neural stem cells (NSCs) via extracellular signal-regulated kinase (ERK) signaling pathway. In another cellular model, melatonin enhanced dopaminergic neuronal differentiation with the effect being potentially brought out by the increased production of brain-derived neurotropic factor (BDNF) and glial cell line-derived neurotrophic factor (GDNF) in the NSCs. Studies indicate Melatonin and Valproic acid work synergistically.

### 4.) Sildenafil Plus N-Acetylcysteine Plus Lithium Carbonate

Vasodilator—Sildenafil Sildenafil produces vasodilation of vascular beds in the brain, lungs, heart and penis by increasing the stability of cyclic guanosine 3′-5′-monophosphate (cGMP). Increased stability of cGMP effectively raises the intracellular level of cGMP. Elevated cGMP relaxes the smooth muscle of blood vessels via the Nitric Oxide pathway which allows increased blood flow to the cerebral and meningeal arteries. This effect augments the delivery of therapeutic agents and endogenous growth factors to injured areas of the brain. Sildenafil also beneficially affects the following: Control of Neuro-inflammation and Angiogenesis. N-acetylcysteine (NAC) is a glutathione precursor with potent antioxidant, pro-neurogenesis and anti-inflammatory properties and a favorable safety profile. Lithium-mediated neuroprotective and neurogenic effects involve mechanisms highly relevant to the post-stroke population including the increased expression of brain-derived neurotrophic factor (BDNF) and Bcl-2, and inhibition of GSK-3β. In vivo studies also reveal that lithium-treated rats compared to controls had a significant increase in MANF (a form of BDNF) expression in the Prefrontal Cortex and Striatum.

### 5.) Candesartan Plus Nortriptyline Plus Rosiglitazone

Candesartan is an angiotensin receptor blocker antihypertensive drug that enhances cerebral blood flow through vasodilation and cerebral vascular density. It is also supportive of neural stem cell proliferation and reduced neuro-inflammation. Nortriptyline is a tricyclic antidepressant that enhances neurogenesis and mitochondrial function. It is also supportive of restorative sleep. Rosiglitazone is a thiazolidinedione drug originally meant to work as an insulin sensitizer. It is an effective reducer of neuroinflammation, promotes neurogenesis and protects axonal growth.

### 6. Sildenafil Plus Curcumin Plus Acetyl-L-Carnetine

Sildenafil produces vasodilation of vascular beds in the brain, lungs, heart and penis by increasing the stability of cyclic guanosine 3′-5′-monophosphate (cGMP). Increased stability of cGMP effectively raises the intracellular level of cGMP. Elevated cGMP relaxes the smooth muscle of blood vessels via the Nitric Oxide pathway which allows increased blood flow to the cerebral and meningeal arteries. This effect augments the delivery of therapeutic agents and endogenous growth factors to injured CNS tissue. Curcumin encapsulated PLGA nanoparticles (Cur-PLGA-NPs) potently induce Neural Stem Cell proliferation (neurogenesis) and neuronal differentiation in vitro and in the hippocampus and subventricular zone of adult rats. Curcumin treatment also shows benefit in decreased chronic neuroinflammation, increased hippocampal neurogenesis, and/or BDNF/Trkb/PI3K/Akt signaling. Dietary supplementation of ALC exerts neuroprotective, neurotrophic, antidepressive and analgesic effects in painful neuropathies. ALC also has antioxidant and anti-apoptotic activity. Moreover, ALC exhibits positive effects on mitochondrial metabolism

### 7. Alzheimer's Treatment

In developing compounds 100 suitable to review for Alzheimer's treatment it has been proposed in the present invention to implement two separate drugs that reduce inflammation 22 as well as two separate drugs or agents that augment bioenergetics 18. This approach leads to a number of promising possibilities.

### 8. Parkinson's Treatment

In developing compounds 100 suitable to review for Parkinson's treatment it has been proposed in the present invention to implement agent combinations that that primarily reduce inflammation 22 and that augment bioenergetics 18 and remyelination 14. This approach leads to a number of promising possibilities.

### 9. Long Covid Combination Drugs

In developing compounds 100 suitable for treatment of Long-COVID use of the matrix 10 developed three combinations according to the present invention including i) Tadalafil, Clemastine and Cilostazol and ii) Guanfacine, N-Acetylcysteine, Pioglitazone; and iii) Acetyl-L-Carnetine, N-Acetylcysteine Amide, and one of Resveratrol or Pterostilbene.

### 10. Concussion/TBI Drug Combinations

In developing compounds 100 suitable for treatment of concussions and/or TBI the implementation matrix 10 resulted in two combinations according to the present invention including i) Metformin+NAC Amide+Sildenafil and ii) NAC+Cilastozol+Clemastine

### 11 PTSD Treatment

The use of LSD with other components is believed to be well suited for PTSD treatments and thus the present invention has a prospective composition 100 for treatment of PTSD including LSD Citalopram for Neurogenesis and Synaptogenesis; Clemastine for remyelination; and at least one of Metformin, Cilostazol, Sildenafil, Memantine or Pentoxifylline for neuro-inflammation

While this invention has been particularly shown and described with references to the preferred embodiments thereof, it will be understood by those skilled in the art that various changes in form and details may be made therein without departing from the scope of the invention.

What is claimed is:

1. A pharmacotherapeutic protocol enhancing neuro-restoration in the human brain for patients suffering with brain injuries and diseases, wherein the protocol comprises treating the patient with a combinatorial pharmacological composition comprising a pharmacologically effective amount of a first Neuroplasticity PA therapeutic agent exhibiting a mechanism of plasticity in at least one of Neurogenesis, Synaptogenesis, Remyelination, Angiogenesis, Bioenergetics, Inflammation and Apoptosis, and a pharmacologically effective amount of a second Neuroplasticity PA therapeutic agent exhibiting a mechanism of plasticity in at least one of Neurogenesis, Synaptogenesis, Remyelination, Angiogenesis, Bioenergetics, Inflammation and Apoptosis; wherein the mechanism of plasticity of the first Neuroplasticity PA therapeutic agent differs from the mechanism of plasticity of the second Neuroplasticity PA therapeutic agent.

2. The pharmacotherapeutic protocol according to claim 1, wherein the patient is suffering from an injury to the central nervous system comprising at least one of stroke, TBI, PTSD, CTE and Spinal Cord Injury.

3. The pharmacotherapeutic protocol according to claim 2, wherein the first Neuroplasticity PA therapeutic agent comprises one of Telmisartan, Metformin and Cilostazol.

4. The pharmacotherapeutic protocol according to claim 3, wherein the second Neuroplasticity PA therapeutic agent comprises one of Telmisartan, Metformin and Cilostazol and which differs from the first Neuroplasticity PA therapeutic agent, and wherein the combinatorial pharmacological composition further includes a third Neuroplasticity PA therapeutic agent.

5. The pharmacotherapeutic protocol according to claim 1, wherein the mechanism of plasticity of the first Neuroplasticity PA therapeutic agent and the mechanism of plasticity of the second Neuroplasticity PA therapeutic agent both includes reducing Inflammation.

6. The pharmacotherapeutic protocol according to claim 1, wherein the mechanism of plasticity of the first Neuroplasticity PA therapeutic agent and the mechanism of plasticity of the second Neuroplasticity PA therapeutic agent both includes enhancing Neurogenesis.

7. The pharmacotherapeutic protocol according to claim 1, wherein the mechanism of plasticity of the first Neuroplasticity PA therapeutic agent and the mechanism of plasticity of the second Neuroplasticity PA therapeutic agent both includes enhancing Angiogenesis.

8. The pharmacotherapeutic protocol according to claim 1, wherein the mechanism of plasticity of the first Neuroplasticity PA therapeutic agent and the mechanism of plasticity of the second Neuroplasticity PA therapeutic agent both includes enhancing Remyelination.

9. The pharmacotherapeutic protocol according to claim 1, wherein the mechanism of plasticity of the first Neuroplasticity PA therapeutic agent and the mechanism of plasticity of the second Neuroplasticity PA therapeutic agent both includes enhancing Bioenergetics.

10. The pharmacotherapeutic protocol according to claim 1, wherein the patient is suffering from at least one of dementia and movement disorders comprising at least one of Alzheimer's disease, Parkinson's disease and ALS.

11. A pharmacotherapeutic protocol enhancing neuro-restoration in the human brain for patients suffering with stroke, wherein the protocol comprises treating the patient with a combinatorial pharmacological composition comprising a pharmacologically effective amount of a first Neuroplasticity PA therapeutic agent exhibiting a mechanism of plasticity and a route of metabolism and excretion, and a pharmacologically effective amount of a second Neuroplasticity PA therapeutic agent exhibiting a mechanism of plasticity and a route of metabolism and excretion; wherein the mechanism of plasticity and the route of metabolism and excretion of the first Neuroplasticity PA therapeutic agent differs from the mechanism of plasticity and the route of metabolism and excretion of the second Neuroplasticity PA therapeutic agent.

12. The pharmacotherapeutic protocol according to claim 11, wherein the first Neuroplasticity PA therapeutic agent comprises one of Telmisartan, Metformin and Cilostazol.

13. The pharmacotherapeutic protocol according to claim 12, wherein the second Neuroplasticity PA therapeutic agent comprises one of Telmisartan, Metformin and Cilostazol.

14. The pharmacotherapeutic protocol according to claim 13, further including a third Neuroplasticity PA therapeutic agent different from the first and second Neuroplasticity PA therapeutic agents.

15. The pharmacotherapeutic protocol according to claim 14, wherein the third Neuroplasticity PA therapeutic agent comprises one of Telmisartan, Metformin and Cilostazol.

16. A Combinatorial pharmacological composition enhancing neuro-restoration in the human brain for patients suffering with brain injuries and diseases, wherein the combinatorial pharmacological composition comprises a pharmacologically effective amount of a first Neuroplasticity PA therapeutic agent exhibiting a mechanism of plasticity and a route of metabolism and excretion, and a pharmacologically effective amount of a second Neuroplasticity PA therapeutic agent exhibiting a mechanism of plasticity and a route of metabolism and excretion; wherein the mechanism of plasticity and the route of metabolism and excretion of the first Neuroplasticity PA therapeutic agent differs from the mechanism of plasticity and the route of metabolism and excretion of the second Neuroplasticity PA therapeutic agent.

17. The Combinatorial pharmacological composition according to claim 16, wherein the first Neuroplasticity PA therapeutic agent comprises one of Telmisartan, Metformin and Cilostazol.

18. The Combinatorial pharmacological composition according to claim 17, wherein the second Neuroplasticity PA therapeutic agent comprises one of Telmisartan, Metformin and Cilostazol.

19. The Combinatorial pharmacological composition according to claim 18, further including a third Neuroplasticity PA therapeutic agent different from the first and second Neuroplasticity PA therapeutic agents.

20. The Combinatorial pharmacological composition according to claim 19, wherein the third Neuroplasticity PA therapeutic agent comprises one of Telmisartan, Metformin and Cilostazol.
